(19) United States
(12) Patent Application Publication
(10) Pub. No.: US 2013/0184826 A1
(43) Pub. Date: Jul. 18, 2013

(54) BIOABSORBABLE ENCLOSURES

(71) Applicant: Basix Spine LLC, Dana Point, CA (US)

(72) Inventor: Gowriharan Thaiyananthan, Whittier, CA (US)

(73) Assignee: BASIX SPINE LLC, Dana Point, CA (US)

(21) Appl. No.: 13/724,580

(22) Filed: Dec. 21, 2012

Related U.S. Application Data
(60) Provisional application No. 61/578,955, filed on Dec. 22, 2011.

(51) Int. Cl. A61F 2/44

(52) U.S. Cl. CPC A61F 2/442
USPC 623/17.16

(57) ABSTRACT

Provided are bioabsorbable enclosures for use in spinal surgical procedures such as fusion of spinal vertebrae. Also provided are methods for enhancing fusion of spinal vertebrae using bioabsorbable enclosures.

### CROSS-REFERENCE TO RELATED APPLICATIONS

This application claims priority to U.S. Provisional Application No. 61/578,955, filed Dec. 22, 2011, which is herein incorporated in its entirety by reference.

### TECHNICAL FIELD

This document relates to bioabsorbable enclosures which are optionally used to enhance fusion of spinal vertebrae.

### BACKGROUND

Currently there are nearly 500,000 spine lumbar and cervical fusion procedures performed each year in the United States. One of the causes of back pain and disability results from the rupture or degeneration of one or more intervertebral discs in the spine. Surgical procedures, including spinal fusion procedures, are commonly performed to correct problems with displaced, damaged, or degenerated intervertebral discs due to trauma, disease, or aging. Optionally, spinal fusion procedures involve removing some or the all of the diseased or damaged disc, and inserting one or more intervertebral implants into the resulting disc space.

### SUMMARY

Provided are bioabsorbable enclosures for use in spinal surgical procedures such as fusion of spinal vertebrae and lamina replacement to protect neural elements. Also provided are methods for enhancing fusion of spinal vertebrae using bioabsorbable enclosures.

For example, provided is a device for implanting in the spinal region of a subject. The device includes a deformable enclosure, which is at least partially defined by a porous, bioabsorbable material. The enclosure is configured to retain a substance that is positioned in the enclosure for a temporal duration during which at least a portion of the substance interacts with the subject through the porous bioabsorbable material of the enclosure.

Optionally, the deformable enclosure has an opening at one end that is sized for insertion of an interbody fusion device into the enclosure. Optionally, the interbody fusion device is positioned within the enclosure. Optionally, the interbody fusion device is an anterior lumbar interbody fusion cage. Optionally, the interbody fusion device is a posterior lumbar interbody fusion cage. Optionally, the interbody fusion device is an anterior and/or posterior cervical interbody fusion cage. Optionally, the interbody fusion device is a lateral and/or oblique interbody fusion cage.

The substance is optionally selected to enhance fusion of two adjacent vertebrae. Optionally, the substance comprises bone, allograft (e.g., cortical and/or cancellous bone, or mixture thereof), autograft, bone paste, bone putty, a growth factor, a pharmaceutical agent, BMP or combinations thereof.

The porous bioabsorbable material optionally comprises nitrocellulose, vicryl mesh, dexon mesh, amniotic membrane, placental tissue, collagen, bioabsorbable polymer, polyglycolic acid (PGA), polylactic acid (PLA), polyparadioxanone (PDS), polycaprolactone (PCL), Poly(L-lactide), Poly(D-Lactide), Poly(DL-lactide), Polyglycolide, L-lactide/DL-lactide, L-lactide/D-lactide, L-lactide/Glycolide, Lactide/Caprolactone, or DL-lactide/Glycolide.

The enclosure is optionally expandable. For example, the enclosure is optionally configured to expand upon administration of the substance into the enclosure. Optionally, the expanded enclosure can have an elongated shape. The expanded enclosure has a length dimension, a width dimension and a height/thickness dimension. Optionally, the height/thickness of the expanded enclosure is between about 0.5 millimeter (mm) and 25.0 millimeters (mm). For example, the height is optionally 5.0 mm. The width dimension of the expanded enclosure is optionally between about 1.0 millimeters and 50.0 millimeters. For example, the width is optionally about 50.0 mm. The length dimension of the expanded enclosure is optionally between about 15.0 millimeters and 100.0 millimeters.

Optionally, the device is configured for placement in the lateral gutter of a lumbar spinal body. Optionally, the enclosure defines a left and a right elongated portions and a connecting portion between the left and the right elongated portions. The right portion is configured for placement in the right lateral gutter of a lumber spinal body and the left portion is configured for placement in the left lateral gutter of a lumbar spinal body. The connecting portion is configured for placement between adjacent spineous processes of two lumbar spinal bodies. The separation between the lateral edges of the left and right elongated portions at a given right plane across the edges is optionally between about 10 millimeters (mm) and 100 millimeters.

Also provided is a method of enhancing spinal fusion in a subject. An example method includes providing a deformable enclosure that is at least partially defined by a porous, bioabsorbable material. The enclosure is configured to retain a substance, which is administered into the enclosure. The enclosure containing the substance is implanted into the spinal region of the subject. The substance positioned in the enclosure is retained therein for a duration of time, during which at least a portion of the substance interacts with the subject through the porous bioabsorbable material of the enclosure. The interaction between the substance and the subject enhances fusion between spinal bodies, or portions thereof, of the subject.

Optionally, the substance promotes bone growth or bone formation in the subject. The substance is optionally selected from the group consisting of: bone, allograft (e.g., cortical and/or cancellous bone, or mixture thereof), autograft, bone paste, bone putty, a growth factor, a pharmaceutical agent, BMP and combinations thereof.

The example method further comprises positioning an interbody fusion device in the deformable enclosure prior to implantation in the spinal region of the subject. The enclosure containing the interbody fusion device into the spinal region of the subject. For example, the enclosure containing the interbody fusion device is implanted in the intervertebral space between two adjacent vertebrae. Optionally, the interbody fusion device is an anterior lumbar interbody fusion cage. Optionally, the interbody fusion device is a posterior lumbar interbody fusion cage. Optionally, the interbody fusion device is a lateral lumbar interbody fusion cage. Optionally, the interbody fusion device is an anterior and/or posterior cervical interbody fusion cage. Optionally, at least a portion of the enclosure is positioned in a lateral gutter of a spinal body.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A is a schematic illustration of an example anterior lumbar interbody fusion cage.

FIG. 1B is a schematic illustration of the example anterior lumbar interbody fusion cage shown in FIG. 1A in a deformable enclosure, which can be at least partially defined by a porous, bioabsorbable material.

FIG. 2 is a schematic illustration of an example deformable enclosure, which can be at least partially defined by a porous, bioabsorbable material, with a substance for enhancing spinal fusion retained within the enclosure.

FIG. 3A is a schematic illustration of an example deformable enclosure positioned in the right and left lateral gutters of spinal vertebrae L3 and L4 and having a connecting portion between the spinal processes of L3 and L4.

FIG. 3B is a schematic illustration of an example deformable enclosure positioned in the right lateral gutters of spinal vertebrae L3 and L4.

FIG. 4A is a plan view of an example deformable enclosure having left and right elongated portions and a connecting portion.

FIG. 4B is a schematic illustration of the example deformable enclosure of FIG. 4A positioned in the right and left lateral gutters of spinal vertebrae L3 and L4 and having a connecting portion between the spinal processes of L3 and L4.

FIG. 4C is a schematic illustration of the example deformable enclosure of FIGS. 4A and 4B showing the right elongated portion positioned in the right lateral gutters of spinal vertebrae L3 and L4.

Like reference symbols in the various drawings indicate like elements.

### DETAILED DESCRIPTION

Provided are bioabsorbable enclosures for use in spinal surgical procedures such as fusion of spinal vertebrae. Also provided are methods for enhancing fusion of spinal vertebrae using bioabsorbable enclosures.

FIGS. 1A and 1B are schematic illustrations of an example anterior lumbar interbody fusion (ALIF) cage. Such cages, and other cages, including posterior lumbar interbody fusion (PLIF), TILF, and lateral body fusion, cages are used in spinal fusion procedures.

For example, a fusion cage, such as a posterior interbody fusion cage can be used in posterior lumbar interbody fusion (PLIF) procedures where the patient has a damaged disc. Such surgical procedures are known in the art, but steps of an example procedure are described below.

A patient is placed in a kneeling position and the patient's skin is cleansed and sterilized. The term patient and subject are used interchangeably herein. An incision is made in the middle of the lower back. The overlying muscles are moved to the side.

The surgeon optionally confirms the correct vertebrae for the procedure using x-ray imaging. The ligamentum flavum is then separated from the lamina above and removed. Parts of the lamina above and below and of the facet joints on each side are removed to increase access.

Next, tracks for the pedicle screws are prepared and the hard bone surface is removed. A guide track is inserted under x-ray guidance. Its depth and position are checked. Then a thread is tapped into the bone. Next, the pedicle screw is placed. The vertebra surface is prepared for the other pedicle screws, and they are placed.

Next, the spinal cord is retracted to one side and half of the damaged disc is removed. Then the remaining half of the disc is removed. The brackets and rods are placed. The intervertebral space is widened to regain normal intervertebral space height and alignment. Then the vertebrae are separated further and held apart by the metal work. Next, the ends of the vertebra bodies are roughened to assist with bone graft fusion. Then, the cage to hold the vertebral bodies apart is laid. The pedicle screw bolts are adjusted to allow some natural compression of the cage. X-rays are used to check the position. The muscles are replaced, and the wound is closed with sutures.

One skilled in the art will appreciate that there are numerous alternatives to the approach described above, including variations of the posterior surgical approach and anterior approaches. The devices and bioabsorbable enclosures described herein are not limited to any specific surgical approach and can be used to enhance fusion with a variety of approaches chosen be the surgeon.

Referring again to FIG. 1A, the ALIF cage 102 includes a peripheral ring of PEEK. The peripheral ring includes an upper vertebral contact surface 110 and a lower vertebral contact surface 112. The cage 102 is configured for positioning between two adjacent lumbar vertebrae such that the upper surface contacts a vertebral body and the lower surface contacts the adjacent vertebral body. The cage 102 further comprises one or more openings 108 for attachment to an insertion device or tool, which is used to guide the cage 102 into the intervertebral space of two vertebrae to be fused. Optionally, the cage 102 includes a central void 103. Bone graft material is optionally positioned in the central void 103 to enhance fusion of the adjacent vertebrae.

As shown in FIG. 1B, a bioabsorbable enclosure 104 may be positioned over at least a portion of the cage 102. The enclosure 104 is deformable such that it can be positioned over at least a portion of the cage 102. The enclosure 104 is at least partially defined by a porous, bioabsorbable material. For example, the enclosure 104 includes one or more pores 106. The pores 106 can vary in number, shape, and size.

The enclosure 104 is configured to retain a substance that is positioned in the enclosure 104 for a temporal duration during which at least a portion of the substance interacts with the subject through the pores 106 of the bioabsorbable material of the enclosure 104. Thus, the size and/or shape and/or size of the pores 106 can vary depending, for example, on the type substance that is to be retained and/or on the time for which the material is to be retained. For example, larger more numerous pores 106 can be used when the substance is a paste, such a bone paste, with a consistency that tends to remain within the enclosure 104. Similarly, smaller or less numerous pores 106 can be used when the substance is more fluid.

Optionally, the deformable enclosure 104 has an opening at one end that is sized for insertion of an interbody fusion device (i.e., cage 102) into the enclosure. Optionally, as shown in FIG. 1B, the interbody fusion device is positioned within the enclosure 104. Optionally, the interbody fusion device is an anterior lumbar interbody fusion cage. Optionally, the interbody fusion device is a posterior lumbar interbody fusion cage. Optionally, the interbody fusion device is a lateral lumbar interbody fusion cage. Optionally, the interbody fusion device is an anterior and/or posterior cervical interbody fusion cage. Although not shown, the deformable enclosure 104 can also be positioned over a screw, pin or other orthopedic device alone or in addition to a cage 102.

A number of substances can be positioned in the enclosure 104 for temporal retention. The term retention does not imply, or exclude, permanent retention. In fact, the enclosure 104 allows communication between the substance in the enclosure 104 and the body of the patient through the pores 106. Such communication allows for the interaction of the patient's body and the substance in the enclosure 104, for example, by diffusion or dissolution of the substance into the patient's body, such as bodily fluids, over time. In addition to communication through the pores 106, the bioabsorbable enclosure 104 is absorbed by the patient's body over time providing additional opportunity for substance retained in the enclosure 104 to interact with the patient's body.

For example, the substance is optionally selected to enhance fusion of two adjacent vertebrae. Optionally, the substance comprises bone, allograft (e.g. bone allograft including, for example, cortical and/or cancellous bone, or mixture thereof), autograft (e.g. bone autograft), bone paste, bone putty, a growth factor, a pharmaceutical agent, BMP or combinations thereof.

The substance is optionally administered into the enclosure 104 after the cage 102 is positioned in the enclosure 104. For example, the substance can be rubbed or pushed through the pores 106 into the space between the enclosure 104 and the cage 102. The substance, can also, or alternatively, be injected into the enclosure 104, for example, using a standard syringe and needle. In any case, the substance can be positioned in the enclosure 104 surrounding the cage 102, including, for example, in the central void 103 of the cage 102. Once the cage 102 is in the enclosure 104 and the substance is positioned in the enclosure 104 the device can be inserted into the intervertebral space using surgical procedures such as that described above.

Optionally, the enclosure 104 includes an opening that allows for insertion of the cage 102 into the enclosure 104. Typically, the opening is larger than a pore 106. The opening can be positioned such that an introducer tool can be inserted into the holes 108 prior to implanting the device into the intervertebral space.

The porous bioabsorbable material optionally comprises nitrocellulose, vicryl mesh, dexon mesh, amniotic membrane, placental tissue, collagen, bioabsorbable polymer, polyglycolic acid (PGA), polylactic acid (PLA), polyparadioxanone (PDS), polycaprolactone (PCL), Poly(L-lactide), Poly(D-Lactide), Poly(DL-lactide), Polyglycolide, L-lactide/DL-lactide, L-lactide/D-lactide, L-lactide/Glycolide, Lactide/Caprolactone, or DL-lactide/Glycolide.

The bioabsorbable material is absorbed by the body over time. Optionally, the biomaterial is impregnated with a growth factor or other factor to facilitate bone in-growth around the interbody cage 102. As the bioabsorbable material is absorbed, therefore, compounds that enhance bone growth can be released from the material itself, as well as, from the substance temporally retained in the enclosure 104.

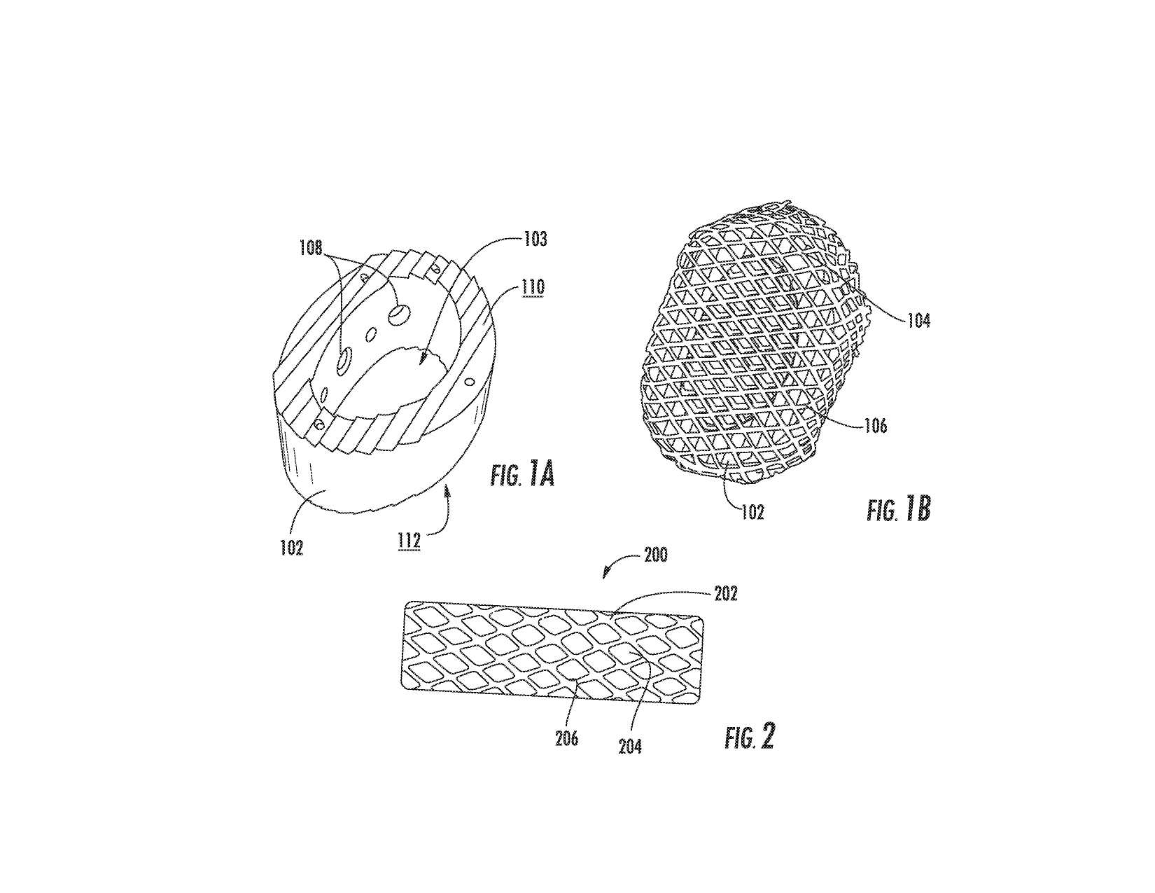

FIG. 2 illustrates another example device 200 for implanting in the spinal region of a patient. The device 200 includes a deformable enclosure 202, which is at least partially defined by a porous, bioabsorbable material. The enclosure 202 is configured to retain a substance 204 that is positioned in the enclosure 202 for a temporal duration during which at least a portion of the substance 204 interacts with the subject through the porous bioabsorbable material of the enclosure 202.

As described with relation to the enclosure 104, the enclosure 202 is at least partially defined by a porous, bioabsorbable material. For example, the enclosure 202 includes one or more pores 206. The pores 206 can vary in number, shape, and size. The enclosure 202 is configured to retain a substance 204 that is positioned in the enclosure 202 for a temporal duration during which at least a portion of the substance 204 interacts with the patient through the pores 206 of the bioabsorbable material of the enclosure 202. Thus, the size and/or shape and/or size of the pores 206 can vary depending, for example, on the type substance 204 that is to be retained and/or on the time for which the material is to be retained. For example, larger more numerous pores 206 can be used when the substance 204 is a paste with a consistency that tends to remain within the enclosure. Similarly, smaller or less numerous pores can be used when the substance 204 is more fluid.

A number of substances 204 can be positioned in the enclosure 202 for temporal retention. The term retention does not imply, or exclude, permanent retention. In fact, the enclosure 202 allows communication between the substance 204 in the enclosure 202 and the body of the patient through the pores 206. Such communication allows for the interaction of the patient's body and the substance 204 in the enclosure 202, for example, by diffusion or dissolution of the substance 204 into the patient's body, such as bodily fluids, over time. In addition to communication through the pores 206, the bioabsorbable enclosure 202 is absorbed by the patient's body over time providing additional opportunity for substance 204 retained in the enclosure 202 to interact with the patient's body.

For example, the substance 204 is optionally selected to enhance fusion of two adjacent vertebrae. Optionally, the substance comprises bone, allograft (e.g. bone allograft including, for example, cortical and/or cancellous bone, or mixture thereof), autograft (e.g. bone autograft), bone paste, bone putty, a growth factor, a pharmaceutical agent, BMP or combinations thereof.

The substance 204 is optionally administered into the enclosure 202. For example, the substance 204 can be rubbed or pushed through the pores 206 into the enclosure 202. The substance 204, can also, or alternatively, be injected into the enclosure 202, for example, using a standard syringe and needle. Once the substance 204 is in the enclosure 202 the device can be inserted into the intervertebral space using surgical procedures such as that described above.

The porous bioabsorbable material optionally comprises nitrocellulose, vicryl mesh, dexon mesh, amniotic membrane, placental tissue, collagen, bioabsorbable polymer, polyglycolic acid (PGA), polylactic acid (PLA), polyparadioxanone (PDS), polycaprolactone (PCL), Poly(L-lactide), Poly(D-Lactide), Poly(DL-lactide), Polyglycolide, L-lactide/DL-lactide, L-lactide/D-lactide, L-lactide/Glycolide, Lactide/Caprolactone, or DL-lactide/Glycolide.

The bioabsorbable material is absorbed by the body over time. Optionally, the biomaterial is impregnated with a growth factor or other factor to facilitate bone in growth and fusion. As the bioabsorbable material is absorbed, therefore, compounds that enhance bone growth can be released from the material itself as well as from the substance temporally retained in the enclosure 202.

The enclosure 202 is optionally expandable. For example, the enclosure 202 is optionally configured to expand upon administration of the substance 204 into the enclosure 202. For example, the substance 204 can be injected into the enclosure 202 to cause expansion of the enclosure 202. As illustrated in FIG. 2, an example enclosure 202 can have an elongated rectangular shape. It is further contemplated that the expanded enclosure can have any other regular or irregular three-dimensional shape including, for example, a square (cube), rectangular (cuboid), cylindrical, spherical, torus, cone, pyramid, and prism.

The expanded enclosure 202 has a length dimension, a width dimension and a height/thickness dimension. Optionally, the height/thickness of the expanded enclosure in the lateral direction is between about 0.5 millimeters (mm) and 25.0 millimeters (mm). For example, the height is optionally, 0.5, 0.6, 0.7, 0.8, 0.9, 1.0, 1.1, 1.2, 1.3, 1.4, 1.5, 1.6, 1.7, 1.8, 1.9, 2.0, 3.0, 4.0, 5.0, 10.0, 15.0, 20.0, or 25.0 mm. The width dimension of the expanded enclosure is optionally between about 1.0 millimeters and 50.0 millimeters. For example, the width is optionally about 1.0, 1.5, 2.0, 2.5, 3.0, 3.5, 4.0, 4.5, 5.0, 5.5, 6.0, 6.5, 7.0, 7.5 8.0 mm, 8.5, 9.0, 10.0, 15.0, 20.0, 25.0, 30.0, 35.0, 40.0, 45.0, or 50.0. The length dimension of the expanded enclosure is optionally between about 15.0 millimeters and 100.0 millimeters. For example, the length is optionally about 15.0, 20.0, 25.0, 30.0, 35.0, 40.0, 45.0, 50.0, 60.0, 70.0, 80.0, 90.0, or 100.0 mm.

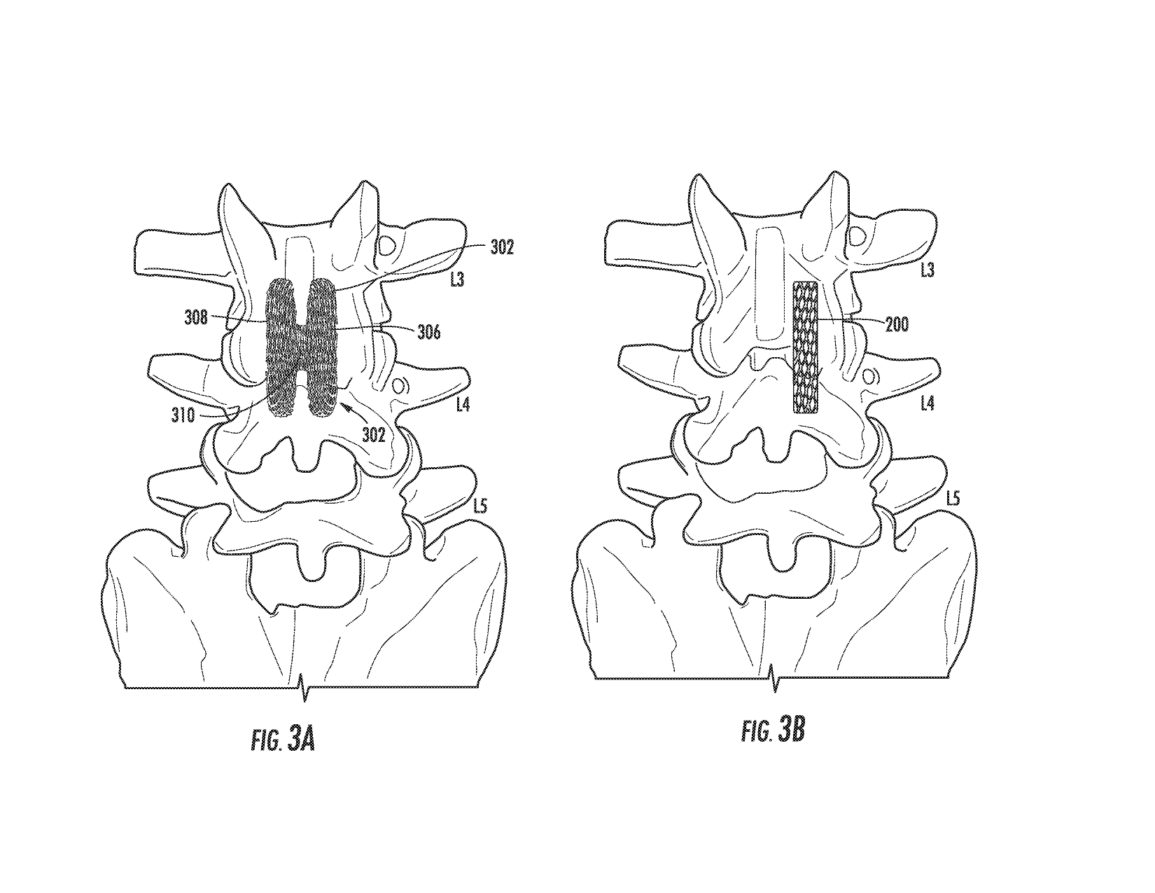

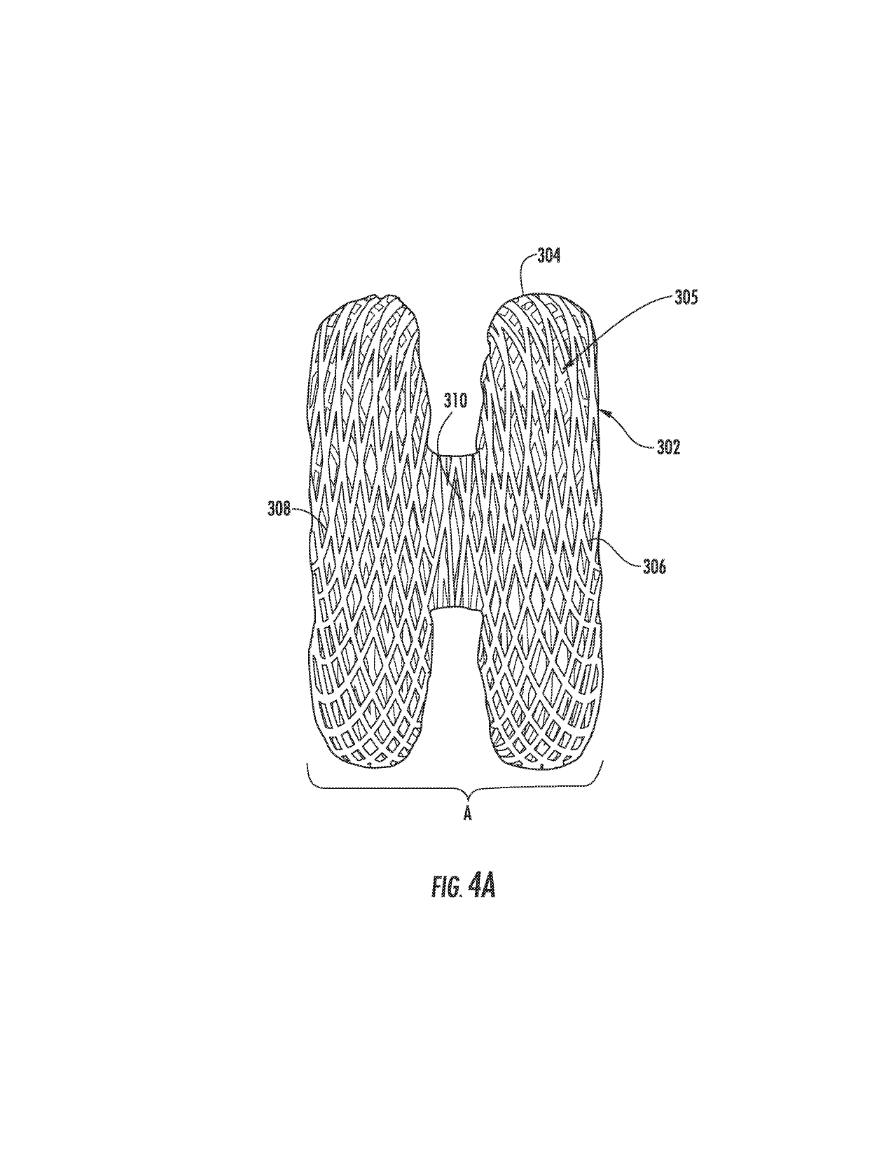

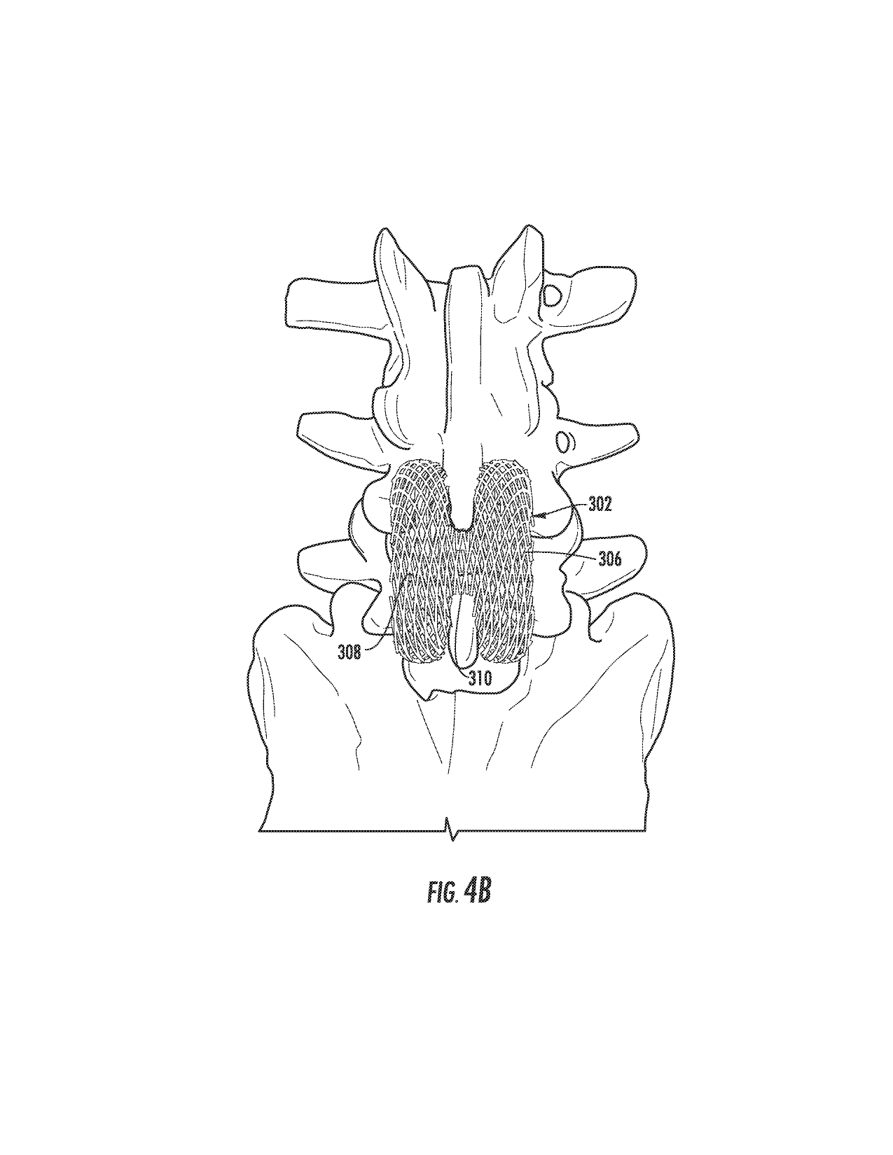

Optionally, the device is configured for placement in the lateral gutter of a lumbar spinal body. Such a placement is shown schematically in FIG. 3B, which shows the device 200 placed in the right lateral gutter of the L3 and L4 vertebrae. Therefore, the device 200 can be used, for example, in posterolateral gutter spine fusion surgery. In an example, the large back muscles that attach to the transverse processes are elevated to create a bed to lay the device 200, including the substance 204, in. Standard decortications methods are optionally used to decorticate the cortical bone on the facet joints and transverse processes. The back muscles are laid back over the bone graft, which creates tension to hold the device 200 in place. Over time, the interaction of the substance 204 and/or enclosure 202 enhance fusion of adjacent vertebrae. A similar procedure can be followed for placement of a device 200 in the left gutter.

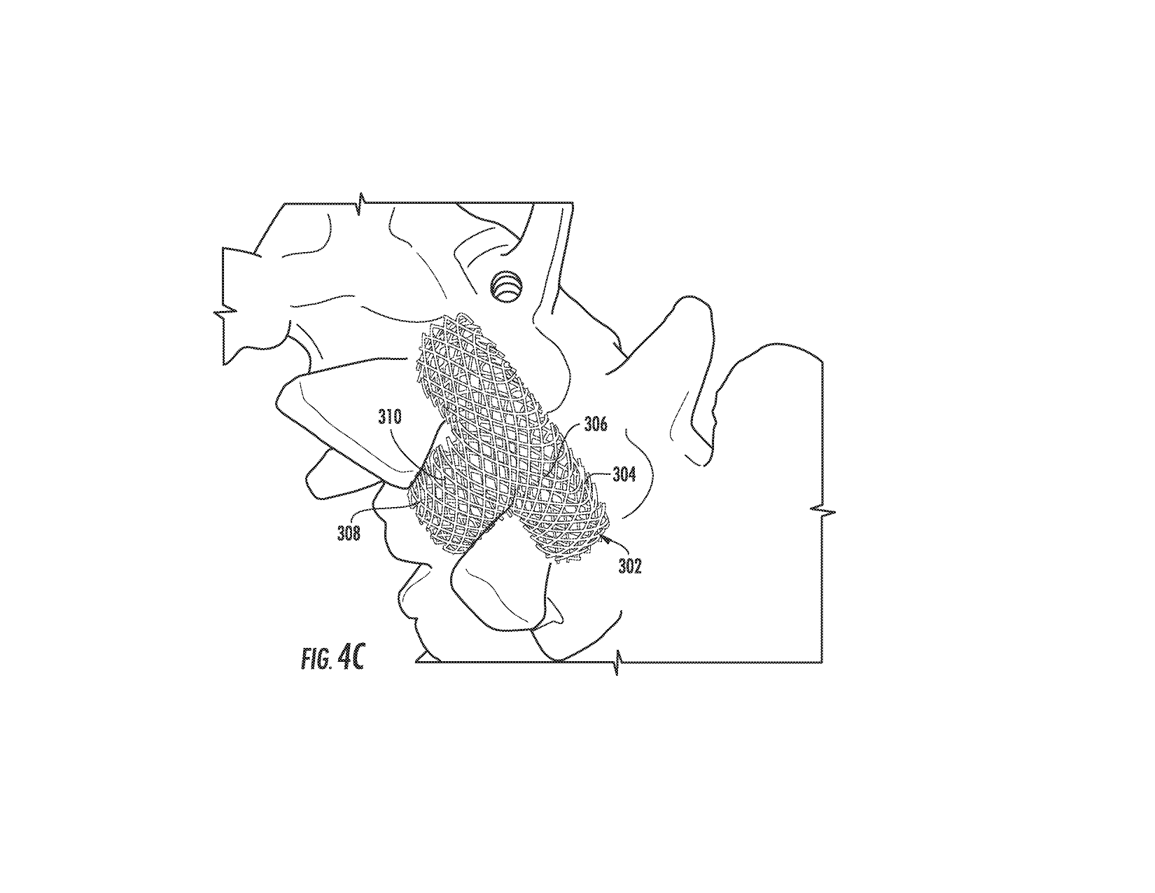

Referring now to FIGS. 3A and 4A-4C, another example interbody fusion device includes an “H-shaped” enclosure 302. The enclosure 302 defines a left 308 and a right 306 elongated portions and a connecting portion 310 between the left and the right elongated portions 308 and 306. Optionally, the right portion 306 can configured for placement in the right lateral gutter of a lumber spinal body and the left portion 308 can be configured for placement in the left lateral gutter of a lumbar spinal body. The connecting portion 310 can be configured for placement between adjacent spineous processes of two lumbar spinal bodies. Optionally, the device 200 can be configured for placement in the in a posterior lateral element of a cervical spine. For example, the left and right elongated portions 308 and 306 can be configured for placement in the posterior lateral element. As illustrated in FIG. 4A, the separation (A) between the lateral edges of the left and right elongated portions 308 and 306 at a given right plane across the edges is optionally between about 10 millimeters (mm) and 100 millimeters. For example, the separation is optionally 10.0, 15.0, 20.0, 21.0, 22.0, 23.0, 24.0, 25.0, 26.0, 27.0, 28.0, 29.0, 30.0, 40.0, 50.0, 60.0, 70.0, 80.0, 90.0, or 100.0 mm.

As described with relation to the devices 104 and 200, the “H-shaped” enclosure 302 is deformable and at least partially defined by a porous, bioabsorbable material 304. For example, the enclosure 302 includes one or more pores 305. The pores 305 can vary in number, shape, and size.

The enclosure 302 is configured to retain a substance that is positioned in the enclosure 302 for a temporal duration during which at least a portion of the substance interacts with the patient through the pores 305 of the bioabsorbable material of the enclosure. Thus, the size and/or shape and/or size of the pores 305 can vary depending, for example, on the type substance that is to be retained and/or on the time for which the material is to be retained. For example, larger more numerous pores 305 can be used when the substance is a paste with a consistency that tends to remain within the enclosure. Similarly, smaller or less numerous pores 305 can be used when the substance is more fluid.

A number of substances can be positioned in the enclosure 302 for temporal retention. The term retention does not imply, or exclude, permanent retention. In fact, the enclosure 302 allows communication between the substance in the enclosure 302 and the body of the patient through the pores 305. Such communication allows for the interaction of the patient's body and the substance in the enclosure 302, for example, by diffusion or dissolution of the substance into the patient's body, such as bodily fluids, over time. In addition to communication through the pores 305, the bioabsorbable enclosure 302 is absorbed by the patient's body over time providing additional opportunity for substance retained in the enclosure 302 to interact with the patient's body.

For example, the substance is optionally selected to enhance fusion of two adjacent vertebrae. Optionally, the substance comprises bone, allograft (e.g. bone allograft including, for example, cortical and/or cancellous bone, or mixture thereof), autograft (e.g. bone autograft), bone paste, bone putty, a growth factor, a pharmaceutical agent, BMP or combinations thereof.

The substance is optionally administered into the enclosure 302. For example, the substance can be rubbed or pushed through the pores 305 into the enclosure 302. The substance, can also, or alternatively, be injected into the enclosure 302, for example, using a standard syringe and needle. Once the substance is in the enclosure 302 the device can be inserted into the intervertebral space using surgical procedures such as that described above.

The porous bioabsorbable material optionally comprises nitrocellulose, vicryl mesh, dexon mesh, amniotic membrane, placental tissue, collagen, bioabsorbable polymer, polyglycolic acid (PGA), polylactic acid (PLA), polyparadioxanone (PDS), polycaprolactone (PCL), Poly(L-lactide), Poly(D-Lactide), Poly(DL-lactide), Polyglycolide, L-lactide/DL-lactide, L-lactide/D-lactide, L-lactide/Glycolide, Lactide/Caprolactone, or DL-lactide/Glycolide.

The bioabsorbable material is absorbed by the body over time. Optionally, the biomaterial is impregnated with a growth factor or other factor to facilitate bone in growth and fusion. As the bioabsorbable material is absorbed, therefore, compounds that enhance bone growth can be released from the material itself as well as from the substance temporally retained in the enclosure 302.

The enclosure 302 is optionally expandable. For example, the enclosure 302 is optionally configured to expand upon administration of the substance into the enclosure 302. For example, the substance can be injected into the enclosure 302 to cause expansion of the enclosure 302. Optionally, one or more of the right and left elongate portions 308 and 306 and the connecting portion 310 are separately expandable.

The left and right elongate portions enclosure each have a length dimension, a width dimension and a height/thickness dimension. Optionally, the height/thickness of the expanded left and/or right elongate portions 308 and 306 is between about 0.5 millimeter (mm) and 25.0 millimeters (mm). For example, the height is optionally, 0.5, 0.6, 0.7, 0.8, 0.9, 1.0, 1.1, 1.2, 1.3, 1.4, 1.5, 1.6, 1.7, 1.8, 1.9, 2.0, 5.0, 10.0, 15.0, 20.0, or 25.0 mm. The width dimension of each of the expanded right and left elongate portions 308 and 306 is optionally between about 1.0 millimeters and 50.0 millimeters. For example, the width is optionally about 1.0, 1.5, 2.0, 2.5, 3.0, 3.5, 4.0, 4.5, 5.0, 5.5, 6.0, 6.5, 7.0, 7.5, 8.0, 8.5, 9.0, 10.0, 15.0, 20.0, 25.0, 30.0, 35.0, 40.0, 45.0, or 50.0 mm. As outlined above, and illustrated in FIG. 4A, the separation (A) between the lateral edges of the left and right elongated portions 308 and 306 is optionally between about 10 millimeters and 100 millimeters. For example, the separation is optionally 10.0, 15.0, 20.0, 21.0, 22.0, 23.0, 24.0, 25.0, 26.0, 27.0, 28.0, 29.0, 30.0, 40.0, 50.0, 60.0, 70.0, 80.0, 90.0, or 100.0 mm. The length dimension of the expanded enclosure is optionally between about 15.0 millimeters and 100.00 millimeters. For example, the length is optionally about 15.0, 20.0, 25.0, 30.0, 35.0, 40.0, 45.0, 50.0, 60.0, 65.0, 70.0, 75.0, 80.0, 85.0, 90.0, 95.0, or 100.0 mm.

A number of embodiments of the invention have been described. Nevertheless, it will be understood that various modifications may be made without departing from the spirit and scope of the invention. Accordingly, other embodiments are within the scope of the following claims.

Disclosed are materials, systems, devices, compositions, and components that can be used for, can be used in conjunction with, can be used in preparation for, or are products of the disclosed methods, systems and devices. These and other components are disclosed herein, and it is understood that when combinations, subsets, interactions, groups, etc. of these components are disclosed that while specific reference of each various individual and collective combinations and permutations of these components may not be explicitly disclosed, each is specifically contemplated and described herein.

For example, if a method is disclosed and discussed each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed systems or devices. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the materials for which they are cited are hereby specifically incorporated by reference in their entireties.

What is claimed is:

1. A device for implanting in the spinal region of a subject, comprising:
a deformable enclosure at least partially defined by a porous, bioabsorbable material;
wherein the enclosure is configured to retain a substance that is positioned in the enclosure for a duration of time, during which at least a portion of the substance interacts with the subject through the porous bioabsorbable material of the enclosure.

2. The device of claim 1, wherein the deformable enclosure has an opening at one end that is sized for the insertion of an interbody fusion device into the enclosure.

3. The device of claim 2, wherein an interbody fusion device is positioned within the enclosure.

4. The device of claim 3, wherein the substance comprises one or more of bone, allograft, autograft, bone paste, bone putty, a growth factor, a pharmaceutical agent, BMP or combinations thereof.

5. The device of claim 4, wherein the interbody fusion device is an anterior lumbar interbody fusion cage.

6. The device of claim 4, wherein the interbody fusion device is a posterior lumbar interbody fusion cage.

7. The device of claim 4, wherein the interbody fusion device is a lateral lumbar fusion device

8. The device of claim 4, wherein the interbody fusion device is an anterior cervical interbody fusion cage.

9. The device of claim 4, wherein the interbody fusion device is a posterior cervical interbody fusion cage.

10. The device of claim 1, wherein the porous bioabsorbable material comprises nitrocellulose, vicryl mesh, dexon mesh, amniotic membrane, placental tissue, collagen, bioabsorbable polymer, polyglycolic acid (PGA), polylactic acid (PLA), polyparadioxanone (PDS), polycaprolactone (PCL), Poly(L-lactide), Poly(D-Lactide), Poly(DL-lactide), Polyglycolide, L-lactide/DL-lactide, L-lactide/D-lactide, L-lactide/Glycolide, Lactide/Caprolactone, or DL-lactide/Glycolide.

11. The device of claim 10, wherein the bioabsorbable material comprises a factor that facilitates bone growth

12. The device of claim 12, wherein the factor that facilities bone growth is a growth factor.

13. The device of claim 1, wherein the enclosure expandable.

14. The device of claim 13, wherein the enclosure is configured to expand upon administration of the substance into the enclosure.

15. The devise of claim 14, wherein the expanded enclosure defines an H-shaped expanded enclosure.

16. The device of claim 14, wherein the expanded enclosure has a length dimension, a width dimension and a height dimension.

17. The device of claim 16, wherein the height of the expanded enclosure is between about 0.5 millimeter (mm) and 25.0 millimeters (mm).

18. The device of claim 17, wherein the width dimension of the expanded enclosure is between about 1.0 millimeters and 50.0 millimeters.

19. The device of claim 18, wherein the length dimension of the expanded enclosure is between about 15.0 millimeters and 100.0 millimeters.

20. The device of claim 1, wherein the device is configured for placement in the lateral gutter of a lumbar spinal body.

21. The devise of claim 1, wherein the device is configured for placement in a posterior lateral element of a cervical spine.

22. The device of claim 1, wherein the enclosure defines a left and a right elongated portions and a connecting portion between the left and the right elongated portions.

23. The device of claim 22, wherein the right portion and the right portion is configured for placement in a posterior lateral element of the cervical spine.

24. The device of claim 22, wherein the right portion is configured for placement in the right lateral gutter of a lumber spinal body.

25. The device of claim 24, wherein the left portion is configured for placement in the left lateral gutter of a lumbar spinal body.

26. The device of claim 25, wherein connecting portion is configured for placement between adjacent spineous processes of two lumbar spinal bodies.

27. The device of claim 25, wherein the separation between the lateral edges of the left and right elongated portions at a given right plane across the edges is between about 10 millimeters and 100 millimeters.

28. A method of enhancing spinal fusion in a subject, comprising:
providing a deformable enclosure that is at least partially defined by a porous, bioabsorbable material, wherein the enclosure is configured to retain a substance;
administering the substance into the enclosure; and
implanting the enclosure into the spinal region of the subject, wherein the substance positioned in the enclosure is retained therein for a duration of time, during which at least a portion of the substance interacts with the subject through the porous bioabsorbable material of the enclosure and wherein the interaction between the substance and the subject enhances fusion between spinal bodies, or portions thereof, of the subject.

29. The method of claim 22, wherein the substance promotes bone growth or bone formation in the subject.

30. The method of claim 23, wherein the substance comprises one or more of bone, allograft, autograft, bone paste, bone putty, a growth factor, a pharmaceutical agent, BMP or combinations thereof.

31. The method of claim 25, further comprising positioning an interbody fusion device in the deformable enclosure.

32. The method of claim 25, further comprising implanting the enclosure containing the interbody fusion device into the spinal region of the subject.

33. The method of claim 26, wherein the interbody fusion device is an anterior lumbar interbody fusion cage.

34. The method of claim 26, wherein the interbody fusion device is a posterior lumbar interbody fusion cage.

35. The method of claim 24, wherein at least a portion of the enclosure is positioned in a lateral gutter of a spinal body.
